(19) United States
(12) Patent Application Publication
(10) Pub. No.: US 2004/0260060 A1
(43) Pub. Date: Dec. 23, 2004

(54) Constructs modified downstreams of the initiation codon for recombinant protein

(76) Inventor: Laurent Chevalet, Cuvat (FR)

Correspondence Address: Norman H Stepno, Burns Doane Swecker & Mathis, PO Box 1404, Alexandria, VA, 22313-1404, US

(21) Appl. No.: 10/311,976

(22) PCT Filed: Jun. 21, 2001
(86) PCT No.: PCT/FR01/01952

(30) Foreign Application Priority Data
Jun. 22, 2000 (FR) 00/08002

(51) Int. Cl. C07K 14/195; C07H 21/04

(52) U.S. Cl. 530/350; 435/69.1; 435/320.1; 435/252.3; 536/23.7

(57) ABSTRACT

The invention concerns a construct for expressing a gene coding for a recombinant protein of interest placed under the control of a tryptophan operon (Ptrp) in a prokaryotic cell, comprising directly downstream of the initiation codon a nucleic sequence SEQ ID N° 1 and downstream of said sequence a multiple cloning cassette designed to receive the gene coding for said recombinant protein of interest, at least nucleic acids of the nucleic sequence SEQ ID N° 1 being mutated or deleted so as to enable overexpression of said recombinant protein. The invention also concerns a vector containing such a construct, a prokaryotic host cell transformed by said vector, as well as a method for producing a recombinant protein of interest using the inventive construct.

The invention relates to a construct for the expression of a gene encoding a recombinant protein of interest placed under the control of the tryptophan operon Ptrp, in a prokaryotic host cell, which comprises, directly downstream of the initiation codon, a nucleic acid sequence of sequence SEQ ID No. 1 and, downstream of this sequence, a multiple cloning cassette intended to receive the gene encoding said recombinant protein of interest, at least one of the nucleotides of the sequence SEQ ID No. 1 being mutated or deleted so as to allow overexpression of said recombinant protein. The invention also relates to a vector containing such a construct, to a prokaryotic host cell transformed with said vector, and also to a method for producing a recombinant protein of interest using a construct according to the invention.

The ability of biotechnologists to clone a gene in short periods of time, to express it in the form of a biologically active protein and then to create variants thereof in order to establish sequence/function relationships has made it possible to propose a wide range of recombinant proteins for medical or research purposes. Many human diseases are now treated or avoided because of the availability of molecules derived from biotechnology in pure form and at an acceptable cost (K. Koths, Current Opinion in Biotechnology, 6, 681-687, 1995).

Bacterial cells are preferred hosts for the expression of recombinant proteins because they have limited nutrient requirements while at the same time being capable of reaching high growth densities, but also because they have been the subject, in the past, of many investigations which have led to the generation of mutants of interest and of varied plasmid expression systems. Among bacteria, Escherichia coli (E. coli) is the most commonly used and most thoroughly characterized organism, judging by the abundant literature relating the expression therein of proteins of prokaryotic or eukaryotic origin. However, not all proteins are expressed therein with the same efficiency due to difficulties which may occur at various levels: transcription of the gene of interest, translation, post-translational events affecting what becomes of the molecule in the cytoplasmic or periplasmic environment of the bacterium (S. C. Makrides, Microbiological Reviews, 60, 512-538, 1996).

In order to be translated efficiently, a messenger RNA must contain a sequence specifying binding of the bacterial ribosome and allowing initiation of translation. This sequence, called ribosome binding site (RBS), is located in a region covering the initiating codon. Statistical analysis of bacterial mRNA initiation domains reveals the existence of a 34 nucleotide window, the sequence of which differs from a random distribution (L. Gold, Annual Review of Biochemistry, 57, 199-233, 1988). This sequence, ranging from position −20 to position +13 of the mRNA if position +1 is attributed to the first nucleotide of the initiating codon, plays the role of RBS by helping the ribosome to distinguish the true initiation domains from all of the “RBS-like” sequences. Many investigations have made it possible to refine knowledge regarding the RBS in order to define some characteristic elements thereof:

i) The Shine-Dalgarno (SD) Sequence:

Since the sequencing of the 3′ end of the 16S ribosomal RNA (J. Shine and L. Dalgarno, Proc. Natl. Acad. Sci. U.S.A., 71, 1342-1346, 1974), the “Shine-Dalgarno” sequence has been defined as the mRNA region positioned 5′ of the initiation codon exhibiting complementarity with the sequence 5′-CCUCCUUA-3′ of the 3′ end of the 16S rRNA. The existence of an interaction between the 16S rRNA and the RBS, mediated by the Shine-Dalgarno sequence, is confirmed by the strong representation of the purine bases A and G in the region [−12; −7] of natural RBSs of E. coli mRNA. This bias is found in a collection of 158 randomized RBSs selected for their ability to promote expression of a reporter gene (D. Barrick et al., Nucleic Acids. Res., 22, 1287-1295, 1994).

ii) The Initiation Codon:

It is the AUG codon which is preferentially used as initiation codon, even though GUG and, to a lesser degree, UUG can occasionally be found) S. Ringquist et al., Molecular Microbiology, 6, 1219-1229, 1992).

iii) The Distance Between the SD Sequence and the Initiation Codon:

An exhaustive study by H. Chen et al. (Nucleic Acids Research, 22, 4953-4957, 1994a) has shown the existence of an optimum distance separating the 3′ end of the Shine-Dalgarno sequence and the initiation codon. Taking the consensus sequence 5′-UAAGGAGGU-3′ as reference SD sequence, the spacing which gives the maximum level of expression is 5 nucleotides. A spacing of between 1 and 9 nucleotides remains favorable, ensuring a level of expression at least equal to 50% of the maximum level.

iv) Other Primary Sequences:

Two pairings are known to be involved in initiating translation: the pairing between mRNA initiation codon and tRNA-fMet, firstly, and the pairing between SD sequence and 16S rRNA 3′ end, secondly. Mutagenesis studies and analysis of atypical mRNAs (in particular mRNAs lacking a leader sequence) have made it possible to identify new sequence elements within the environment of the AUG codon which may contribute to the overall efficiency of the initiation domain. Adenine-rich motifs immediately downstream of the initiation codon are favorable to translation initiation (G. F. E. Scherer et al., Nucleic Acids Research, 8, 3895-3907, 1980; H. Chen et al., Journal of Molecular Biology, 240, 20-27, 1994b). Similarly, the AAA and GCU codons, which are the most common in the second codon position (L. Gold, 1988), have a positive effect on translation, especially when the initiation codon is suboptimal (GUG or UUG) (S.

Ringquist et al., 1992). A sequence identified on the mRNA of the T7 phage 0.3 gene, and named “Downstream Box” (DB) due to its position downstream relative to the initiation codon, is another translation-promoting element (M. L. Sprengart et al., Nucleic Acids Research, 18, 1719-1723, 1990). This 12 nucleotide sequence exhibits complementarity with nucleotides 1469-1483 of 16S rRNA, and it is found in similar forms on translation initiation domains of several highly expressed E. coli and bacteriophage genes (M. L. Sprengart et al., 1990). This “Downstream Box” allows translation initiation even in the absence of SD sequence (M. L. Sprengart et al., The EMBO Journal, 15, 665-674, 1996). Recent results indicate that, contrary to the hypothesis initially put forward, the DB sequence could act via a mechanism other than pairing with the 1469-1483 region of 16S rRNA (M. O'Connor et al., Proc. Natl. Acad. Sci. U.S.A., 96, 8973-8978, 1999).

v) Secondary Structures:

The sequence of the mRNA in proximity to the SD region may influence translational efficiency via the formation of secondary structures. M. H. de Smit and J. van Duin (Journal of Molecular Biology, 235, 173-184, 1994) show that intramolecular pairings on the mRNA can be harmful to correct translation by competing with the mRNA/rRNA pairing, all the more so the weaker the complementarity of the SD region with the 16S rRNA. In the same way, it has been shown that the expression of prochymosin in E. coli is dependent on the composition of the region connecting SD to the initiation codon: a sequence which limits secondary structures promotes accessibility of the RBS to the ribosome and leads to high translational efficiency (G. Wang et al., Protein Expression and Purification, 6, 284-290, 1995).

Given the importance of the translation initiation step on the yield of expression of recombinant proteins, many studies have been carried out with the aim of optimizing the RBS region of bacterial expression vectors. An intuitive approach has first consisted in placing the complete consensus SD region (UAAGGAGGU) upstream of genes of interest (G. Jay et al., Proc. Natl. Acad. Sci. U.S.A., 78, 5543-5548, 1981). More systematically, D. M. Marquis et al. (Gene, 42, 175-183, 1986) have placed this sequence downstream of various promoters and at a varying distance (5 to 9 nucleotides) from the initiation codon. With the IL-2 gene as a model, the results indicate that an SD/AUG spacing of 6 nucleotides is optimal for almost all the promoters tested. In a comparative study between the consensus SD sequence and the SD sequence of the lacZ gene, W. Mandecki et al. (Gene, 43, 131-13, 1986) have, however, noted that the consensus SD sequence gives greater expression in vitro but expression which is 2- to 2.5-fold weaker than that of lacZ in vivo. Whole RBS regions derived from phage genes with their own SD sequence have also proved to be superior to the consensus SD sequence for the expression of proteins of various origins (plants, mammalian cells, bacteria) (P. O. Olins et al., Gene, 73, 227-235, 1988). Using the tryptophan promoter, K. Curry and C. S. C. Tomich (DNA, 7, 173-179, 1988) have compared the efficiency of the consensus SD sequence with that present naturally in Ptrp. Their results indicate a very strong dependency with respect to the gene of interest studied, coming to the conclusion that it is impossible to construct an optimal vector which functions for all heterologous genes. M. K. Olsen et al. (Journal of Biotechnology, 9, 179-190, 1989), themselves also working with the tryptophan promoter and the consensus SD sequence, have obtained very high levels of expression (20 to 30% of total proteins) for various heterologous proteins (growth hormones, TNF) by enriching the sequences flanking the SD region with A and T nucleotides.

Similar results had been described previously by H. A. De Boer et al. (DNA, 2, 231-235, 1983) who noted the positive effect of A and T bases placed downstream of the SD region in the context of the hybrid promoter Ptrp/PlacUV5 expressing α-interferon.

All these results were obtained in the context of experiments in which a limited number of parameters were taken into account. Aware of the large number of factors, known or unknown, with an influence on the initiation of translation, and especially of the a priori not insignificant role of the interactions between factors which are not taken into account in iterative approaches, some authors subsequently tried to select optimal synthetic RBSs, in vivo, from large-size random libraries. B. S. Wilson et al. (BioTechniques, 17, 944-952, 1994) thus screened a repertoire of sequences degenerate on 16 positions upstream of the initiation codon, within an expression cassette containing the β-lactamase gene under the control of the lac promoter/operator. Such an approach made it possible to identify original sequences expressing the β-lactamase with a 3-fold greater efficiency. With another gene encoding an scFv, the level of overexpression relative to the original RBS is approximately 2-fold.

In view of these results, it is established that RBS regions which are described as being optimal are always described as such in a particular context in which both the sequence of the gene of interest and the sequence of the mRNA leader region, which itself depends on the type of promoter used, are involved. The tryptophan promoter (B. P. Nichols and C. Yanofsky, Methods in Enzymology, 101, 155-164, 1983) is one of the major systems used in recombinant protein expression (D. G. Yansura and D. J. Henner, Methods in Enzymology (Anonymous Academic Press, Inc., San Diego, Calif.) 54-60, 1990; D. G. Yansura and S. H. Bass, Methods in Molecular Biology, 62, 55-62, 1997), but its RBS has never been the subject of systematic optimization using an approach based on the screening of random sequences.

It is important for the biotechnologist wishing to develop industrial-scale methods to have tools which guarantee maximum expression irrespective of the protein of interest. As a result, there is a great deal of interest in any enhancement which makes it possible to optimize the expression of recombinant proteins, whether the enhancements are introduced via the host strain, via the expression vector, via the method of culturing and expression or via any combination of these factors.

More particularly, the present invention demonstrates the advantage, in terms of translational efficiency, of novel nucleotide sequences, carried by an expression vector, in the ribosome binding site (RBS) region, downstream of the tryptophan promoter (Ptrp).

Using degenerate oligonucleotides introduced upstream of the initiation codon, and then selecting clones overexpressing the chloramphenicol acetyltransferase (CAT) reporter gene, the applicant sought novel optimized RBS sequences. In searching for optimized sequences upstream of the initiation codon, it was discovered, most surprisingly, that the nucleic acid sequence located directly downstream of the initiation codon could be mutated or deleted so as to overexpress recombinant proteins. The sequences thus obtained exhibit a characteristic of enhancement with respect to the expression of various genes of interest of diverse origins.

In addition, a major current problem with regard to current constraints of quality is to obtain a recombinant protein which is as pure as possible, i.e. with a minimum number of amino acids grafted upstream or downstream of the recombinant protein, these being amino acids originating from the construct used. When the nucleic acid sequence located between the initiation codon and the first cloning site has deletions so as to overexpress recombinant proteins, this problem is also solved by the present invention.

A subject of the present invention is thus a construct for the expression of a gene encoding a recombinant protein of interest placed under the control of the tryptophan operon promoter Ptrp, in a prokaryotic host cell, comprising, directly downstream of the initiation codon, a nucleic acid sequence of sequence SEQ ID No. 1 and, downstream of this sequence, a multiple cloning cassette intended to receive the gene encoding said recombinant protein of interest, characterized in that at least one of the nucleotides of the sequence SEQ ID No. 1 is mutated or deleted so as to allow overexpression of said recombinant protein.

It is all the more surprising to obtain overexpression by virtue of the subject of the invention since the prior art teaches the use of this sequence SEQ ID No. 1 without modification. To this effect, mention may in particular be made of patents U.S. Pat. No. 5,714,589, U.S. Pat. No. 5,468,845, U.S. Pat. No. 5,418,135, U.S. Pat. No. 4,891,310, U.S. Pat. No. 4,789,702, WO 88/09344, U.S. Pat. No. 4,738,921 and EP 0 212 532, which teach the use of the sequence SEQ ID No. 1 downstream of the initiation codon for the expression of proteins of interest.

The expression “recombinant protein of interest” is intended to denote all proteins, polypeptides or peptides obtained by genetic recombination and able to be used in fields such as that of human or animal health, of cosmetology, of animal nutrition, of the agro industry or of the chemical industry. Among these proteins of interest, mention may in particular be made, but without being limited thereto, of:

a cytokine and in particular an interleukin, an interferon, a tissue necrosis factor and a growth factor and in particular a hematopoietic growth factor (G-CSF, GM-CSF), a human growth hormone or insulin, a neuropeptide;

a factor or cofactor involved in clotting and in particular factor VIII, von Willebrand factor, antithrombin III, protein C, thrombin and hirudin;

an enzyme and in particular trypsin, a ribonuclease and β-galactosidase;

an enzyme inhibitor such as α1-antitrypsin and viral protease inhibitors;

a protein capable of inhibiting the initiation or progression of cancers, such as expression products of tumor suppressor genes, for example the P53 gene;

a protein capable of stimulating an immune response or an antigen, such as, for example, Gram-negative bacterial membrane proteins, or active fragments thereof, in particular Klebsiella OmpA proteins or the human respiratory syncytial virus protein G;

a monoclonal antibody which may or may not be humanized or an antibody fragment such as an scFv;

a protein capable of inhibiting a viral infection or its development, for example the antigenic epitopes of the virus in question or modified variants of viral proteins, capable of competing with the native viral proteins;

a protein liable to be contained in a cosmetic composition, such as substance P or a superoxide dismutase;

a dietary protein and in particular an alicament;

an enzyme capable of directing the synthesis of chemical or biological compounds, or capable of degrading certain toxic chemical compounds; or else

any protein having a toxicity with respect to the microorganism which produces it, in particular if this microorganism is the E. coli bacterium, such as, for example, the HIV-1 virus protease, the ECP protein, “eosinophil cationic protein”, or poliovirus proteins 2B and 3A.

The expression “nucleic acid sequence of sequence SEQ ID No. 1, at least one of the nucleic acids of which is mutated or deleted so as to allow overexpression of said recombinant protein” is intended to mean any sequence which comprises a deletion or a mutation of at least one nucleotide of the sequence SEQ ID No. 1, which allows overexpression of the recombinant protein compared to the expression of said recombinant protein obtained using the unmodified sequence SEQ ID No. 1.

The term “deletion” is intended to mean the removal of one or more nucleotides at one or various nucleotide sites of the sequence SEQ ID No. 1. The resulting sequence is shortened compared to the original one.

The term “mutation” is intended to mean the replacement of a nucleic acid with another (A with C, G or T; C with A, G or T; G with A, C or T; T with A, C or G). The resulting sequence has the same length as the original one.

The overexpression, i.e. the fact of obtaining an expression greater than that obtained without the modification downstream of the initiation codon, can be determined in particular using one of the following methods:

i) migrating the total proteins of the bacterium, by SDS-PAGE, and revealing the recombinant protein by staining with Coomassie Blue or by Western blotting;

ii) assaying the recombinant protein by a method involving a specific antibody (Elisa);

iii) enzymatic assaying if the recombinant protein possesses a catalytic activity.

Preferentially, method ii), details of which are given in example III, is used.

The expression “multiple cloning cassette” is intended to mean a nucleotide sequence containing one or more restriction sites, which sites can be used in steps of cloning the gene of interest downstream of the initiation codon.

Preferentially, said at least nucleotide of the sequence SEQ ID No. 1 is deleted so as to allow overexpression of said recombinant protein.

The invention also relates to a construct according to the invention in which said at least nucleotide which is mutated or deleted, preferentially deleted, is located on the fragment of sequence SEQ ID No. 2 of the sequence SEQ ID No. 1.

Another subject of the invention concerns the constructs in which said at least nucleotide which is mutated or deleted, preferentially mutated, is located on the codon GTA and/or on the codon GCA and/or on the codon CTG of the sequence SEQ ID No. 1.

In a preferred embodiment of the invention, said sequence SEQ ID No. 1, at least one of the nucleotides of which is mutated or deleted, has the nucleotide A at least at position 1, 2 and 3.

In a preferred embodiment of the invention, at least one of the nucleotides, and preferentially all the nucleotides, located between the nucleic acid sequence of sequence SEQ ID No. 1 and the multiple cloning cassette intended to receive the gene encoding said recombinant protein of interest are deleted.

In another even more preferred embodiment of the invention, said sequence SEQ ID No. 1, at least one of the nucleic acids of which is mutated or deleted, and all the nucleotides of which that are located between the nucleic acid sequence of sequence SEQ ID No. 1 and the multiple cloning cassette are completely deleted, such that the initiation codon is directly upstream of the multiple cloning cassette.

In a preferred embodiment of the invention, the constructs contain a nucleic acid sequence directly upstream of the initiation codon, which sequence is chosen from the sequences of sequence SEQ ID No. 3, SEQ ID No. 4, SEQ ID No. 5, SEQ ID No. 6, SEQ ID No. 7, SEQ ID No. 8, SEQ ID No. 9 and SEQ ID No. 10.

The invention comprises a construct according to the invention, characterized in that the prokaryotic host cell is a gram-negative bacterium, preferably belonging to the species E. coli.

Another subject of the invention concerns a vector containing a construct as defined above, as it does a prokaryotic host cell, preferably belonging to the species E. coli, transformed with such a vector.

A subject of the present invention is also a method for producing a recombinant protein of interest in a host cell using a construct as defined above.

A subject of the present invention is also a method for producing a recombinant protein of interest according to the invention, in which said construct is introduced into a prokaryotic host cell, preferentially via a vector as defined above.

Preference is given to a method for producing a recombinant protein of interest according to the invention, characterized in that it comprises the following steps:

a) cloning a gene of interest into a vector according to the invention;

b) transforming a prokaryotic cell with a vector containing a gene encoding said recombinant protein of interest;

c) culturing said transformed cell in a culture medium which allows expression of the recombinant protein; and

d) recovering the recombinant protein from the culture medium or from said transformed cell.

The invention also comprises the use of a construct, of a vector or of a prokaryotic host cell according to the present invention, for producing a recombinant protein.

Finally, the invention relates to the use of a recombinant protein, for preparing a medicinal product intended to be administered to a patient requiring such a treatment, characterized in that said recombinant protein is produced using a method for producing a recombinant protein of interest according to the invention.

The following examples and figures are intended to illustrate the invention without in any way limiting the scope thereof.

Legend of the Figures and of the Tables:

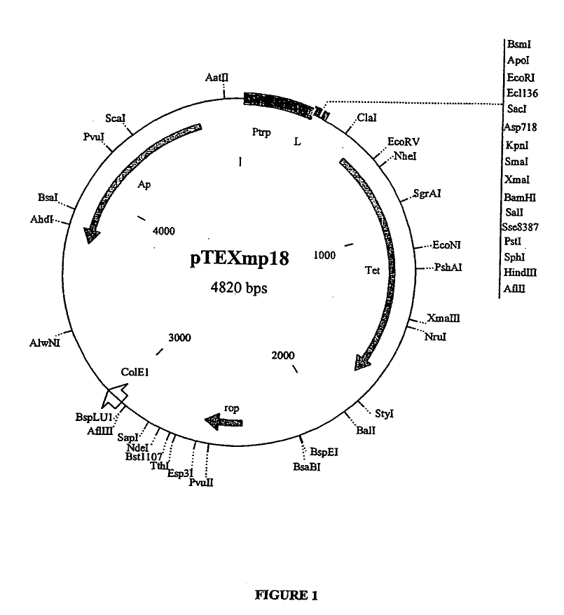

FIG. 1: Map of the plasmid vector pTEXmp18 and sequence SEQ ID No. 39 of the region 1-450 comprising the Ptrp promoter/operator, the TrpL leader region, the mp18 multiple cloning site and the transcription terminator.

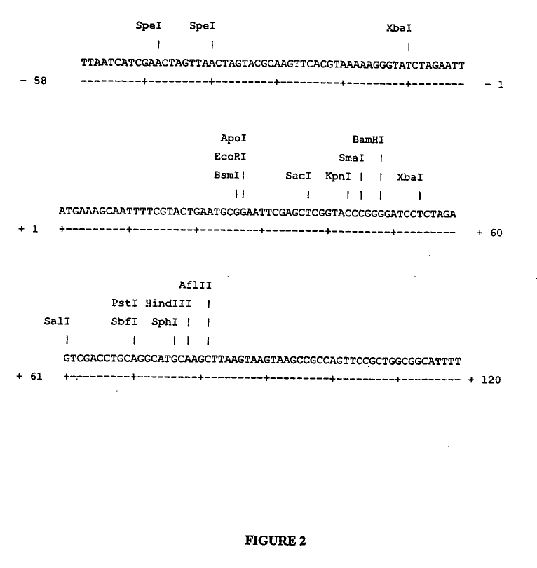

FIG. 2: Restriction map of the RBS (ribosome binding site) region on the vector pTEXmp18 (SEQ ID No. 40).

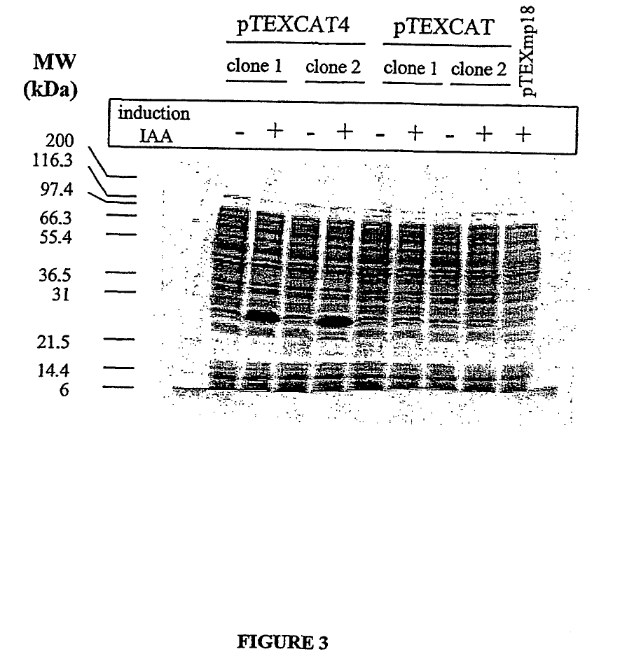

FIG. 3: Estimation on SDS-PAGE gel of the CAT expression in bacteria transformed with the vectors pTEXCAT or pTEXCAT4.

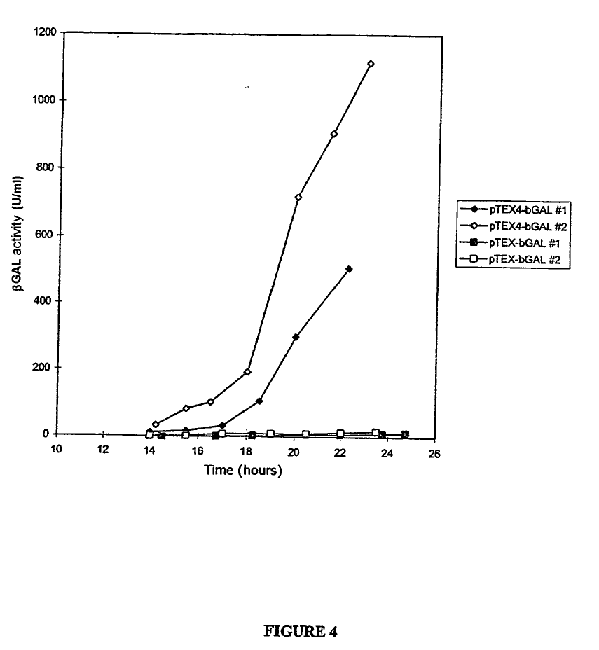

FIG. 4: Comparative study of the expression of β-galactosidase using the vectors pTEX-βGAL and pTEX4-βGAL (kinetics in a fermenter).

### EXAMPLE I

This example illustrates one of the aspects which led to the invention, and in particular the manner in which the library of plasmid vectors carrying the Ptrp tryptophan promoter, and randomly mutated upstream of the initiation codon, is constructed. The vector of origin is described in FIG. 1. It is a plasmid derived from pBR322 (F. Bolivar et al., Gene, 2, 95-113, 1977) into which has been cloned the Ptrp promoter/operator (1-298), followed by the sequence encoding the first 7 amino acids of the E. coli TrpL leader (C. Yanofsky et al., Nucleic Acids Research, 9, 6647-6668, 1981), by a multiple cloning site and by the E. coli trpt transcription terminator (C. Yanofsky et al., 1981). The 3′ portion of Ptrp in pTEXmp18 differs from the natural sequence by the presence of an XbaI cloning site upstream of the ATG initiation codon (see FIG. 2) and by a longer spacing between SD and the initiation codon.

In order to allow the selection of vectors modified in their RBS portion, the chloramphenicol acetyl-transferase (CAT) reporter gene is cloned at the EcORI and PstI sites of pTEXmp18. For this, the coding sequence of the cat gene is amplified by PCR using the oligonucleotides CATfor and CATrev, the sequences of which are:

| | |
|---|---|
| CATfor: 5′-CCGGAATTCATGGAGAAAAAAATCACTGG-3′ | (SEQ ID No. 11) |
| EcoRI | |
| CATrev: 5′-AAACTGCAGTTACGCCCCGCCCTG-3′ | (SEQ ID No. 12) |
| PstI | |

The PCR reaction is carried out using the phagemid pBC-SK (Stratagene, La Jolla, Calif., USA) as matrix. The amplification product is loaded onto agarose gel and purified according to the GeneClean method (Bio101, La Jolla, Calif.). The cloning of the insert into pTEXmp18 is verified, after transformation into E. coli, by the appearance of colonies which develop on dishes of LB agar medium (J. Sambrook et al., Molecular cloning. A laboratory manual, 2nd edition. Plainview, N. Y.: Cold Spring Harbor Laboratory Press, 1989) in the presence of 30 μg/ml of chloramphenicol. The sequence of the insert is confirmed by automatic sequencing using the “Dye Terminator” kit and the DNA sequencer 373A (Perkin Elmer Applied Biosystems, Foster City, Calif.). The vector obtained is called pTEXCAT.

Insertion of RBS having a degenerate sequence upstream of the initiation codon is carried out by ligation of synthetic oligonucleotides at the SpeI and EcORI sites of the vector pTEXCAT. The region ranging from the SpeI site to the EcORI site respectively at positions −49 and +28 (see FIG. 2) is deleted by enzymatic digestion and replaced with a heteroduplex formed by two partially degenerate synthetic oligonucleotides hybridized to one another. Two pairs of oligonucleotides are used, involving respectively the oligonucleotides RanSD1/RanSD2 and RanSD3/RanSD4, the sequences of which are:

| | |
|---|---|
| 5′CTAGTTAACTAGTACGCAAGTTCACGTAAANNNNNNNNNNNNNNNNATG | (SEQ ID No. 13) |
| AAAGCAATTTTCGTACTGAATGCGG-3′ | |
| RanSD2: | |
| 5′AATTCCGCATTCAGTACGAAAATTGCTTTCATNNNNNNNNNNNNNNNNTT | (SEQ ID No. 14) |
| TACGTGAACTTGCGTACTAGTTAA-3′ | |
| RanSD3: | |
| 5′CTAGTTAACTAGTACGCAAGTTCACGTAAATRRRRRRRNNNNNNATGAAA | (SEQ ID No. 15) |
| GCAATTTTCGTACTGAATGCGG-3′ | |
| RanSD4: | |
| 5′AATTCCGCATTCAGTACGAAAATTGCTTTCATNNNNNNYYYYYYYATTTA | (SEQ ID No. 16) |
| CGTGAACTTGCGTACTAGTTAA-3′. | |

The four oligonucleotides were synthesized by MWG Biotech (Ebersberg, Germany) under conditions ensuring equimolar distribution of the bases for each degeneracy. The pair RanSD1/RanSD2 introduces complete degeneracy (N=mixture of the 4 nucleotides A, C, T, G) on the 16 nucleotides preceding the ATG codon. The number of combinations (416, i.e. approximately 4.3×10⁹) allows RBSs to be screened which are optimized both from the point of view of their Shine-Dalgarno (SD) sequence and the sequence located between the SD region and the initiation codon, and also in the SD-ATG spacing. This library will be named (N₁₆) in the remainder of the text. The pair RanSD3/RanSD4 introduces complete degeneracy on 6 nucleotides preceding the ATG and partial degeneracy on the 7 nucleotides upstream. The exclusive use of purines (R=A or G) on the positive strand and of pyrimidines on the complementary strand (Y=C or T) promotes the representation of sequences of the Shine-Dalgarno type at an optimal distance (6 nucleotides) from the ATG codon. This second library is named (R₇N₆).

The linearization of the vector pTEXCAT and the gel purification thereof, the hybridization of the oligonucleotides in pairs, the ligation of the heteroduplexes to the linearized vector pTEXCAT and the transformation into E. coli of the library thus constituted are carried out according to the conditions described by J. Sambrook et al. (1989). Conventionally, 100 fmol of vector and 1 000 fmol of insert are added to a ligation reaction in the presence of T4 ligase in a final volume of 15 μl. The reaction is carried out overnight at 16° C. Electrocompetent TOP10 bacteria (50 μl) are then transformed by electroporation with 3 μl of the ligation mixture, under the conditions recommended by the manufacturer (Invitrogen, Carlsbad, Calif.). The transformation mixture is plated out on LB agar dishes containing 200 μg/ml of ampicillin, giving rise, after incubation for 16 hours at 37° C., to the appearance of transformed colonies.

### EXAMPLE II

The libraries are screened based on the hypothesis that the clones overexpressing the CAT enzyme will have increased resistance to chloramphenicol. This is validated by the experiment the results of which are given in table 1 below.

TABLE 1
Chloramphenicol resistance of TOP10 x pTEXCAT
bacteria in the presence or absence of IAA

| | Chloramphenicol concentration (μg/ml) | | | | | | | |
|---|---|---|---|---|---|---|---|---|
| | 0 | 200 | 300 | 400 | 500 | 600 | 700 | 800 |
| IAA = 0 | 85 | 91 | 74 | 73 | 47 | 0 | 0 | 0 |
| | +++ | +++ | ++ | + | +/− | − | − | − |
| IAA = 25 | 75 | 65 | 76 | 53 | 71 | 1 | 0 | 0 |
| μg/ml | +++ | +++ | +++ | ++ | + | − | − | − |

The numbers (upper row) indicate the number of colonies counted after incubation for 18 h at 37° C., each medium having been seeded with approximately 100 cells.

The index of the lower row is a qualitative criterion of colony growth (−=absence of growth to +++=maximum growth).

These results show that TOP10 E. coli bacteria (Invitrogen, Carlsbad, Calif.) transformed with the vector pTEXCAT and plated out on dishes containing various concentrations of chloramphenicol develop, between 300 and 600 μg/ml of chloramphenicol, more strongly in the presence of 3-β indole acrylic acid (IAA), a tryptophan analog which acts as an inducer via a Ptrp derepression effect (R. Q. Marmorstein and P. B. Sigler, The Journal of Biological Chemistry, 264, 9149-9154, 1989). This implies that clones which overproduce CAT due to an optimized RBS region may either develop more rapidly than the wild-type population at a chloramphenicol concentration lower than the MIC (mininimum inhibitory concentration), or develop in the presence of chloramphenicol concentrations which are lethal for the wild-type population.

### EXAMPLE III

This example illustrates the selection of clones from the libraries constructed according to the description of example 1. The libraries obtained in the form of layers of colonies on dishes of LB agar+ampicillin are taken up in sterile water so as to reconstitute a suspension with an optical density (OD) at 580 nm in the region of 1. In accordance with the results of example 2, this suspension is plated out on LB agar dishes containing lethal doses of chloramphenicol (600, 700, 800 and 900 μg/ml) in a proportion of 100 μl of suspension per Petri dish. The dishes are incubated at 37° C. and the appearance of resistant colonies is observed, verifying at the same time that dishes seeded using a suspension of TOP10 bacteria transformed with the wild-type pTEXCAT vector do not give any growth.

The resistant colonies are isolated and subcultured several times on the selection medium in order to confirm their resistance phenotype. The clones selected at this stage are then subjected to a series of analyses: (i) extraction of the plasmid (Qiagen kit, Hilden, Germany) and sequencing of the region covering the RBS, (ii) culturing in Erlenmeyer flasks with induction by IAA and then estimation of the level of CAT expression by ELISA assay, (iii) electrophoresis, by SDS-PAGE, of the total proteins extracted from the preceding cultures and staining with Coomassie Blue to visualize total intracellular proteins. The clones are sequenced using the Dye Terminator kit on an ABI 373A sequencer (Perkin Elmer Applied Biosystems, Foster City, Calif.). The cultures in Erlenmeyer flasks are prepared by seeding 25 ml of TSBY (30 g/l tryptic soy broth (DIFCO)+5 g/l yeast extract (Difco)) medium+8 mg/l tetracycline with a colony on a dish or with a bacterial suspension stored at −80° C. Each preculture is incubated on a platform shaken at 200 rpm, at 37° C. overnight. A fraction is transferred into 50 ml of the same medium so as to reach an initial optical density equal to 1. To induce the CAT protein, 25 mg/l of IAA are added to the medium, which is then shaken under the same conditions for 5 hours. A fraction of the suspension (3×1 ml diluted to OD=0.1) is centrifuged and the cells are stored at −20° C. for assaying the CAT by ELISA (CAT ELISA kit, Roche Diagnostics, Basel, Switzerland). The remainder of the biomass is recovered by centrifugation at 10 000 g, 4° C. for 15 minutes. The biomass is taken up in TEL buffer (25 mM Tris, 1 mM EDTA, 500 μg/ml lysozyme, pH 8) in a proportion of 5 ml per g of wet biomass. The cells are lysed by sonication (VibraCell sonicator equipped with a microprobe, Sonics & Materials, Danbury, Conn.). One ml of the resulting suspension is centrifuged for 5 min at 12 000 rpm. The pellet is taken up with 200 μl of TEL, to give the insoluble (I) fraction. The supernatant is marked “S”. The total proteins contained in the I and S fractions are analyzed by electrophoresis under denaturing conditions (SDS-PAGE) and staining with Coomassie Blue.

Table 2 below indicates the various RBS sequences obtained after screening the two libraries (N₁₆) and (R₇N₆). After alignment in the GenBank and EMBL nucleotide databases, we can conclude that none of the 16-nucleotide ((N₁₆) strategy) or 13-nucleotide ((R₇N₆) strategy) sequences located immediately upstream of the AUG codon in the various isolated clones has been described to date.

TABLE 2

Novel RBS sequences isolated using one of the
strategies (N₁₆) or (R₇N₆)

| | | |
|---|---|---|
| CLONE | STRATEGY | REGION SD - L PEPTIDE (*) |

| | | |
|---|---|---|
| PTEXCAT | — | |
| SEQ ID No. 19 | | AAGGGUAUCUAGAAUUAUGAAAGCAAUUUUCGUACUGAAUGCGGAAUUC |
| SEQ ID No. 20 | | M K A I F V L N A E F |
| PTEXCAT4 | (N₁₆) | |
| SEQ ID No. 21 | | GGGCCGGUUUCUUAUUAUGAAAGCAAUUUUCGUACCGAAUGCGGAAUUC |
| SEQ ID No. 22 | | M K A I F V P N A E F |
| pTEXCAT1′ | (R₇N₆) | |
| SEQ ID No. 23 | | UGGGAGGGUCAAUUAUGAAACCAAUUUUCGUACUGAAUGCGGAAUUC |
| SEQ ID No. 24 | | M K P I F V L N A E F |
| pTEXCAT2′ | (R₇N₆) | |
| SEQ ID No. 25 | | UAAAGGAACCAUAUAUGAAA***************AAUGCGGAAUUC |
| SEQ ID No. 26 | | M K * * * * * N A E F |
| pTEXCAT3′ | (R₇N₆) | |
| SEQ ID No. 27 | | UAGGAAAGAUAACGAUGAAAGCAAUUUUCGCACUGAAUGCGGAAUUC |
| SEQ ID No. 28 | | M K A I F A L N A E F |
| pTEXCAT5′ | (R₇N₆) | |
| SEQ ID No. 29 | | UGAGGAGAAGACAGAUGAAAGCAAU*********GAAUGCGGAAUUC |
| SEQ ID No. 30 | | M K A M * * * N A E F |
| pTEXCAT9′ | (R₇N₆) | |
| SEQ ID No. 31 | | UGAGGAGAGUAAUCAUGAAAGCA***************GCGGAAUUC |
| SEQ ID No. 32 | | M K A * * * * * A K F |

(*) Each nucleotide sequence (messenger RNA) comprises the mutated region downstream of the initiation codon. The reference sequence of the vector pTEXCAT appears in the first line of the table. The nucleic acid sequences upstream and downstream of the initiation codon of the vectors are represented in this table after transcription in the form of RNA. At the 3′ end of these sequences, only the first two codons of the multiple cloning site are represented, namely GAAUUC.

Thus, it was observed, most surprisingly, that the clones described in table 2 have mutations in the RBS region located immediately downstream of the AUG codon.

The clones pTEXCAT4, pTEXCAT1′ and pTEXCAT3′ carry a point mutation affecting an amino acid of the N-terminal portion of the encoded protein (respectively Leu7Pro, Ala3Pro and Val6Ala). The other clones carry larger rearrangements: pTEXCAT2′, pTEXCAT5′ and pTEXCAT9′ have deletions which induce, respectively, the loss of the regions Ala3Leu7, Ile4Leu7 and Ile4Asn8. Given that the random analysis of 10 clones of the (N₁₆) library, selected on ampicillin (i.e. without chloramphenicol selection pressure), shows no modification in the region encoding the TrpL peptide (data not shown), it is deduced therefrom that the mutations in TrpL observed on the clones selected for their ability to express CAT play a role in the expression. Thus, we demonstrate the following original property: the expression of recombinant proteins is positively affected by mutations downstream of the initiation codon.

FIG. 3 presents an SDS-PAGE analysis of the total proteins of bacteria transformed with pTEXCAT or pTEXCAT4. It shows confirmation of the overproducing characteristic of the vector pTEXCAT4 since a major protein which migrates at the position expected for CAT (28 kDa) is clearly demonstrated in IAA-induced extracts, whereas the extracts of the vector pTEXCAT, obtained under the same induction conditions, reveal only a band of low intensity.

In order to exclude the possibility that the overproduction is caused by modifications of the vector outside the SpeI-EcORI portion, the vector pTEXCAT4 was reconstructed in vitro from pTEXCAT by SpeI-EcORI digestion and ligation of a duplex formed by the following two phosphorylated oligonucleotides:

| | |
|---|---|
| SDopt4-f: | |
| 5′CTAGTTAACTAGTACGCAAGTTCACGTAAAACGGAGAAACCCCCCAATGA | (SEQ ID No. 17) |
| AAGCAATTTTCGTACCGAATGCGG-3′ | |
| SDopt4-r: | |
| 5′AATTCCGCATTCGGTACGAAAATTGCTTTCATTGGGGGGTTTCTCCGTTTT | (SEQ ID No. 18) |
| ACGTGAACTTGCGTACTAGTTAA-3′. | |

The resulting vector, marked pTEXCAT-SD4, was then transformed into E. coli TOP10 and compared with pTEXCAT4 in terms of CAT enzyme expression potential. The results obtained indicate that the levels of expression of pTEXCAT4 and pTEXCAT-SD4 are comparable to one another and significantly greater than pTEXCAT. This substantiates the hypothesis that the enhancement of expression observed with the clones claimed in this patent application is indeed caused specifically by the sequences located between the SpeI and EcORI sites.

In order to demonstrate the specificity of the mutated or deleted sequences located directly downstream of the initiation codon, the leucine CTG at the seventh position of the wild-type vector pTEXCAT was replaced with a proline CCG, to give the vector pTEXCAT-L7P. The proline CCG at the seventh position of the vector pTEXCAT4 was replaced with a leucine CTG, to give the vector pTEXCAT4-P7L. The results of this experiment appear in table 3 below.

TABLE 3
Comparison between the levels of expression
given by the vectors pTEXCAT, pTEXCAT4, pTEXCAT-L7P and
pTEXCAT4-P7L

| Vector | Level of CAT expression |
|---|---|
| PTEXCAT | 1 |
| pTEXCAT4 | 128 ± 0.7 |
| pTEXCAT-L7P | 119 ± 2 |
| pTEXCAT4-P7L | 1.9 ± 0.1 |

The results (mean±standard deviation) were obtained on two independent experiments. The level 1 is arbitrarily assigned to the vector PTEXCAT.

These results demonstrate that the mutation downstream of the initiation codon is by itself responsible for the overexpression, since this mutation reintroduced into the wild-type vector makes it possible to obtain the same overexpression.

### EXAMPLE IV

This example shows that the effect of overexpression of the novel sequences described is not limited to the reporter gene used to select them, but is transposed to other genes once these genes are functionally linked to them on the same vector. To this effect, the CAT gene of the vectors pTEXCAT and pTEXCAT4 was replaced with the sequence of the lacZ gene encoding E. coli β-galactosidase. The cloning was carried out by amplifying the lacZ sequence by PCR using the vector pβGAL-basic (Clontech, Palo Alto, Calif.) and then inserting this sequence downstream of trpL at the unique BsmI and HindIII sites, to give, respectively, the vectors pTEX-βGAL and pTEX4-βGAL.

The two vectors were transformed into the E. coli strain ICONE 200 (French patent application FR 2 777 292 published on Oct. 15, 1999) for the purpose of culturing in a fermenter with β-galactosidase expression kinetics being followed.

Conventionally, the recombinant bacteria ICONE 200× pTEX-βGAL and ICONE 200× pTEX4-βGAL were cultured in 200 ml of complete medium (30 g/l tryptic soy broth (DIFCO), 5 g/l yeast extract (DIFCO)) overnight at 37° C. The cell suspension obtained was transferred sterilely into a fermenter (Chemap model CF3000, volume 3.5 l) containing 1.8 liters of the following medium (concentrations for 2 liters of final culture): 90 g/l glycerol, 5 g/l (NH₄)₂SO₄, 6 g/l KH₂PO₄, 4 g/l K₂HPO₄, 9 g/l Na3-citrate.2H₂O, 2 g/l MgSO₄.7H₂₀, 1 g/l yeast extract, trace elements, 0.06% antifoaming agent, 8 mg/l tetracycline, 200 mg/l tryptophan. The pH is set at 7.0 by adding aqueous ammonia. The dissolved oxygen level is maintained at 30% of saturation by servo-control of the rate of shaking and then of the aeration rate by measuring dissolved O₂. When the optical density of the culture reaches a value of between 0.30 and 40, induction is carried out by adding 25 mg/l of IAA (Sigma, St Louis, Mo.). A kinetic analysis of the optical density of the culture (OD at 580 nm) and of the intracellular β-galactosidase activity was carried out. The level of β-galactosidase activity is estimated by colorimetric assaying by mixing 30 μl of sample (fraction “S”, see example 3), 204 μl of buffer (50 mM Tris-HCl, pH 7.5-1 mM MgCl₂) and 66 μl of ONPG (4 mg/ml in 50 mM Tris-HCl, pH 7.5). The reaction mixture is incubated at 37° C. The reaction is stopped by adding 500 μl of 1M Na₂CO₃. The OD at 420 nm, related to the incubation time, is proportional to the β-galactosidase activity present in the sample. Since E. coli ICONE 200 has a complete deletion of the lac operon, the β-galactosidase activity measured is due only to the expression of the plasmid lacZ gene.

The results of this comparative study indicate that, in two independent experiments, the vector pTEX4-βGAL gives a level of β-galactosidase activity approximately 50 times greater than pTEX-βGAL (FIG. 4). We deduce therefrom that the original sequence isolated in the RBS region of the vector pTEXCAT4 potentiates the expression not only of the CAT protein, but also of other proteins such as, by way of example, β-galactosidase. Based on this example, we can conclude that other proteins of biotechnological interest may be advantageously expressed using one of the vectors according to the invention, by introducing their coding sequence downstream of the mutated or deleted sequences according to the invention.

### EXAMPLE V

Comparison between the levels of expression given by the vectors pTEXwt (which is not part of the invention) and the vectors pTEX9′, pTEX10′, pTEX11′ and pTEX121.

The vectors pTEX10′, pTEX11′ and pTEX12′, are derived from the vector pTEX9, but also comprise additional mutations, as indicated in table 4 below:

TABLE 4

Comparison between the levels of expression
given by the vectors pTEXwt, pTEX9′, pTEX10′,
pTEX11′ and pTEX12′

| | | |
|---|---|---|
| | | CAT |
| Vector | REGION SD - L PEPTIDE (*) | expression |

| | | |
|---|---|---|
| PTEXwt | | |
| SEQ ID No. 19 | AAGGGUAUCUAGAAUUAUGAAAGCAAUUUUCGUACUGAAUGCGGAAUUC | 1 |
| SEQ ID No. 20 | M K A I F V L N A E F | |
| PTEX9′ | | |
| SEQ ID No. 31 | UGAGGAGAGUAAUCAUGAAAGCA***************GCGGAAUUC | 249 |
| SEQ ID No. 32 | M K A * * * * * A E F | |
| pTEX10′ | | |
| SEQ ID No. 33 | UGAGGAGAGUAAUCAUGAAAGCA******************GAAUUC | 253 |
| SEQ ID No. 34 | M K A * * * * * * E F | |
| pTEX11′ | | |
| SEQ ID No. 35 | UGAGGAGAGUAAUCAUGAAA*********************GAAUUC | 124 |
| SEQ ID No. 36 | M K * * * * * * * E F | |
| pTEX12′ | | |
| SEQ ID No. 37 | UGAGGAGAGUAAUCAUG************************GAAUUC | 155 |
| SEQ ID No. 38 | M * * * * * * * * E F | |

(*) Each nucleotide sequence (messenger RNA) comprises the mutated region downstream of the initiation codon. The reference sequence of the vector pTEXCAT appears in the first line of the table. The nucleic acid sequences upstream and downstream of the initiation codon of the vectors are represented in this table after transcription in the form of RNA. At the 3′ end of these sequences, only the first two codons of the multiple cloning site are represented, namely GAAUUC.

The methods for determining the expression are those used in the examples above.

These results demonstrate that the deletions downstream of the initiation codon make it possible to obtain an overexpression up to more than 250 times greater than the expression observed using the wild-type vector.

1. A construct for the expression of a gene encoding a recombinant protein of interest placed under the control of the tryptophan operon Ptrp, in a prokaryotic host cell, comprising, directly downstream of the initiation codon, a nucleic acid sequence of sequence SEQ ID No. 1 and, downstream of this sequence, a multiple cloning cassette intended to receive the gene encoding said recombinant protein of interest, characterized in that at least one of the nucleotides of the sequence SEQ ID No. 1 is mutated or deleted so as to allow overexpression of said recombinant protein.

2. The construct as claimed in claim 1, characterized in that at least one of the nucleotides of the sequence SEQ ID No. 1 is deleted.

3. The construct as claimed in claim 1, characterized in that said at least nucleotide which is mutated or deleted is located on the fragment of sequence SEQ ID No. 2 of the sequence SEQ ID No. 1.

4. The construct as claimed in claim 1, characterized in that said at least nucleotide which is mutated or deleted, preferentially mutated, is located on the codon GTA of the sequence SEQ ID No. 1.

5. The construct as claimed in claim 1, characterized in that said at least nucleotide which is mutated or deleted, preferentially mutated, is located on the codon GCA of the sequence SEQ ID No. 1.

6. The construct as claimed in claim 1, characterized in that said at least nucleotide which is mutated or deleted, preferentially mutated, is located on the codon CTG of the sequence SEQ ID No. 1.

7. The construct as claimed in claim 1, characterized in that said sequence SEQ ID No. 1, at least one of the nucleic acids of which is mutated or deleted, has the nucleotide A at least at position 1, 2 and 3.

8. The construct as claimed in claim 1, characterized in that said sequence SEQ ID No. 1 is completely deleted.

9. The construct as claimed in one of claims 1 to 8, characterized in that at least one of the nucleotides, and preferentially all the nucleotides, located between the nucleic acid sequence of sequence SEQ ID No. 1 and the multiple cloning cassette intended to receive the gene encoding said recombinant protein of interest is deleted.

10. The construct as claimed in any one of claims 1 to 9, characterized in that the nucleic acid sequence directly upstream of the initiation codon is chosen from the sequences SEQ ID No. 3 to SEQ ID No. 10.

11. The construct as claimed in any one of claims 1 to 10, characterized in that the prokaryotic host cell is a gram-negative bacterium.

12. The construct as claimed in any one of claims 1 to 11, characterized in that the prokaryotic host cell is E. coli.

13. A vector containing a construct as claimed in any one of claims 1 to 12.

14. A prokaryotic host cell transformed with a vector as claimed in claim 13.

15. The prokaryotic host cell as claimed in claim 14, characterized in that it is E. coli.

16. A method for producing a recombinant protein of interest in a host cell using a construct as claimed in any one of claims 1 to 12.

17. The method for producing a recombinant protein of interest as claimed in claim 16, in which said construct is introduced into a prokaryotic host cell.

18. The method for producing a recombinant protein of interest as claimed in claim 16 or 17, in which said construct is introduced into a prokaryotic host cell via a vector as claimed in claim 13.

19. The method for producing a recombinant protein of interest as claimed in one of claims 16 to 18, characterized in that it comprises the following steps:
a) cloning a gene of interest into a vector as claimed in claim 13;
b) transforming a prokaryotic cell with a vector containing a gene encoding said recombinant protein of interest;
c) culturing said transformed cell in a culture medium which allows expression of the recombinant protein; and
d) recovering the recombinant protein from the culture medium or from said transformed cell.

20. The use of a construct as claimed in one of claims 1 to 12, of a vector as claimed in claim 13 or of a cell as claimed in claim 14 or 15, for producing a recombinant protein.

21. The use of a recombinant protein, for preparing a medicinal product intended to be administered to a patient requiring such a treatment, characterized in that said recombinant protein is produced using a method as claimed in one of claims 16 to 19.


SEQUENCE LISTING (40 sequences)

SEQ ID NO: 1 - DNA, 18 bp, Artificial sequence
  Sequence derived from ribosome binding site of bacterial sequences (RBS).
aaagcaattttcgtactg

SEQ ID NO: 2 - DNA, 9 bp, Artificial sequence
  Sequence derived from ribosome binding site of bacterial sequences (RBS).
tttcgtact

SEQ ID NO: 3 - DNA, 16 bp, Artificial sequence
  Sequence derived from ribosome binding site of bacterial sequences (RBS).
aagggtatctagaatt

SEQ ID NO: 4 - DNA, 16 bp, Artificial sequence
  Sequence derived from ribosome binding site of bacterial sequences (RBS).
gggccggtttcttatt

SEQ ID NO: 5 - DNA, 16 bp, Artificial sequence
  Sequence derived from ribosome binding site of bacterial sequences (RBS).
acggagaaacccccca

SEQ ID NO: 6 - DNA, 14 bp, Artificial sequence
  Sequence derived from ribosome binding site of bacterial sequences (RBS).
tgggagggtcaatt

SEQ ID NO: 7 - DNA, 14 bp, Artificial sequence
  Sequence derived from ribosome binding site of bacterial sequences (RBS).
taaaggaaccatat

SEQ ID NO: 8 - DNA, 14 bp, Artificial sequence
  Sequence derived from ribosome binding site of bacterial sequences (RBS).
taggaaagataacg

SEQ ID NO: 9 - DNA, 14 bp, Artificial sequence
  Sequence derived from ribosome binding site of bacterial sequences (RBS).
tgaggagaagacag

SEQ ID NO: 10 - DNA, 14 bp, Artificial sequence
  Sequence derived from ribosome binding site of bacterial sequences (RBS).
tgaggagagtaatc

SEQ ID NO: 11 - DNA, 29 bp, Artificial sequence
  Primer derived from CAT reporter-gene (Chloroamph,nicol Ac,tyl-Transf,rase).
ccggaattcatggagaaaaaaatcactgg

SEQ ID NO: 12 - DNA, 24 bp, Artificial sequence
  Primer derived from CAT reporter-gene (Chloroamph,nicol Ac,tyl-Transf,rase).
aaactgcagttacgccccgccctg

SEQ ID NO: 13 - DNA, 74 bp, Artificial Sequence
  Degenerated oligonucleotide RanSD1.
ctagttaactagtacgcaagttcacgtaaannnnnnnnnnnnnnnnatgaaagcaattttcgtactgaatgcgg

SEQ ID NO: 14 - DNA, 74 bp, Artificial Sequence
  Degenerated oligonucleotide RanSD2.
aattccgcattcagtacgaaaattgctttcatnnnnnnnnnnnnnnnntttacgtgaacttgcgtactagttaa

SEQ ID NO: 15 - DNA, 72 bp, Artificial Sequence
  Degenerated oligonucleotide RanSD3.
ctagttaactagtacgcaagttcacgtaaatrrrrrrrnnnnnnatgaaagcaattttcgtactgaatgcgg

SEQ ID NO: 16 - DNA, 72 bp, Artificial Sequence
  Degenerated oligonucleotide RanSD4.
aattccgcattcagtacgaaaattgctttcatnnnnnnyyyyyyyatttacgtgaacttgcgtactagttaa

SEQ ID NO: 17 - DNA, 74 bp, Artificial sequence
  Phosphorylated oligonucleotide SDopt4-f.
ctagttaactagtacgcaagttcacgtaaaacggagaaaccccccaatgaaagcaattttcgtaccgaatgcgg

SEQ ID NO: 18 - DNA, 74 bp, Artificial sequence
  Phosphorylated oligonucleotide SDopt4-r.
aattccgcattcggtacgaaaattgctttcattggggggtttctccgttttacgtgaacttgcgtactagttaa

SEQ ID NO: 19 - RNA, 49 nt, Artificial sequence
  Sequence derived from ribosome binding site of bacterial sequences (RBS).
aaggguaucuagaauuaugaaagcaauuuucguacugaaugcggaauuc

SEQ ID NO: 20 - protein, 11 aa, Artificial sequence
  Peptide derived from Escherichia Coli TrpL leader N-terminal sequence
MKAIFVLNAEF

SEQ ID NO: 21 - RNA, 49 nt, Artificial sequence
  Sequence derived from ribosome binding site of bacterial sequences (RBS).
gggccgguuucuuauuaugaaagcaauuuucguaccgaaugcggaauuc

SEQ ID NO: 22 - protein, 11 aa, Artificial sequence
  Peptide derived from Escherichia Coli TrpL leader N-terminal sequence
MKAIFVPNAEF

SEQ ID NO: 23 - RNA, 47 nt, Artificial sequence
  Sequence derived from ribosome binding site of bacterial sequences (RBS).
ugggagggucaauuaugaaaccaauuuucguacugaaugcggaauuc

SEQ ID NO: 24 - protein, 11 aa, Artificial sequence
  Peptide derived from Escherichia Coli TrpL leader N-terminal sequence
MKPIFVLNAEF

SEQ ID NO: 25 - RNA, 32 nt, Artificial sequence
  Sequence derived from ribosome binding site of bacterial sequences (RBS).
uaaaggaaccauauaugaaaaaugcggaauuc

SEQ ID NO: 26 - protein, 6 aa, Artificial sequence
  Sequence derived from ribosome binding site of bacterial sequences (RBS).
MKNAEF

SEQ ID NO: 27 - RNA, 47 nt, Artificial sequence
  Sequence derived from ribosome binding site of bacterial sequences (RBS).
uaggaaagauaacgaugaaagcaauuuucgcacugaaugcggaauuc

SEQ ID NO: 28 - protein, 11 aa, Artificial sequence
  Peptide derived from Escherichia Coli TrpL leader N-terminal sequence
MKAIFALNAEF

SEQ ID NO: 29 - RNA, 38 nt, Artificial sequence
  Sequence derived from ribosome binding site of bacterial sequences (RBS).
ugaggagaagacagaugaaagcaaugaaugcggaauuc

SEQ ID NO: 30 - protein, 8 aa, Artificial sequence
  Peptide derived from Escherichia Coli TrpL leader N-terminal sequence
MKAMNAEF

SEQ ID NO: 31 - RNA, 32 nt, Artificial sequence
  Sequence derived from ribosome binding site of bacterial sequences (RBS).
ugaggagaguaaucaugaaagcagcggaauuc

SEQ ID NO: 32 - protein, 6 aa, Artificial sequence
  Peptide derived from Escherichia Coli TrpL leader N-terminal sequence
MKAAEF

SEQ ID NO: 33 - RNA, 29 nt, Artificial sequence
  Sequence derived from ribosome binding site of bacterial sequences (RBS).
ugaggagaguaaucaugaaagcagaauuc

SEQ ID NO: 34 - protein, 5 aa, Artificial sequence
  Peptide derived from Escherichia Coli TrpL leader N-terminal sequence
MKAEF

SEQ ID NO: 35 - RNA, 26 nt, Artificial sequence
  Sequence derived from ribosome binding site of bacterial sequences (RBS).
ugaggagaguaaucaugaaagaauuc

SEQ ID NO: 36 - protein, 4 aa, Artificial sequence
  Peptide derived from Escherichia Coli TrpL leader N-terminal sequence
MKEF

SEQ ID NO: 37 - RNA, 23 nt, Artificial sequence
  Sequence derived from ribosome binding site of bacterial sequences (RBS).
ugaggagaguaaucauggaauuc

SEQ ID NO: 38 - protein, 3 aa, Artificial sequence
  Peptide derived from Escherichia Coli TrpL leader N-terminal sequence
MEF

SEQ ID NO: 39 - DNA, 445 bp, Artificial sequence
  Sequence derived from plasmidic vector pTEXmp18.
gaattaattcatgctgtggtgtcatggtcggtgatcgccagggtgccgacgcgcatctcgactgcacggtgcaccaatgcttctggcgtcaggcagccatcggaagctgtggtatggctgtgcaggtcgtaaatcactgcataattcgtgtcgctcaaggcgcactcccgttctggataatgttttttgcgccgacatcataacggttctggcaaatattctgaaatgagctgttgacaattaatcatcgaactagttaactagtacgcaagttcacgtaaaaagggtatctagaattatgaaagcaattttcgtactgaatgcggaattcgagctcggtacccggggatcctctagagtcgacctgcaggcatgcaagcttaagtaagtaagccgccagttccgctggcggcattttttttgatcctaacgctcggttgccgcc

SEQ ID NO: 40 - DNA, 178 bp, Artificial sequence
  Sequence derived from plasmidic vector pTEXmp18.
ttaatcatcgaactagttaactagtacgcaagttcacgtaaaaagggtatctagaattatgaaagcaattttcgtactgaatgcggaattcgagctcggtacccggggatcctctagagtcgacctgcaggcatgcaagcttaagtaagtaagccgccagttccgctggcggcatttt